(19) United States
(12) Patent Application Publication
(10) Pub. No.: US 2019/0145894 A1
(43) Pub. Date: May 16, 2019

(54) Determining Characteristics of Enzyme Catalysis

(71) Applicant: Oxford University Innovation Limited, Oxforxd (GB)

(72) Inventors: Omer Dushek, Oxford (GB); Jesse Goyette, Oxford (GB)

(73) Assignee: Oxford University Innovation Limited, Oxford (GB)

(21) Appl. No.: 16/300,265

(22) PCT Filed: May 9, 2017
(86) PCT No.: PCT/GB2017/051286

§ 371 Date: Nov. 9, 2018

(30) Foreign Application Priority Data
May 9, 2016 (GB) 1608058.2

(51) Int. Cl. G01N 21/552; C12Q 1/25; G16C 20/10; G06N 7/00; G16C 10/00; G16B 99/00

(52) U.S. Cl. CPC G01N 21/553; C12Q 1/25; G16C 20/10; G06F 17/13; G16C 10/00; G16B 99/00; G06N 7/00

(57) ABSTRACT

Embodiments of the present invention provide a computer-implemented method of determining characteristics of enzyme catalysis, comprising receiving, from a surface plasmon resonance (SPR) instrument, SPR data indicative of binding of anenzyme and a substrate, and determining one or more characteristics of the enzyme catalysis based on a multi-centre particle density (MPD) model and the SPR data.

### BACKGROUND

Enzymes are macromolecules that catalyse a range of biological reactions, both inside and outside cells. In “tethered” enzymatic reactions a binding domain associated with the enzyme enables its localisation. Examples of tethered enzymatic reactions of this sort are prevalent in cell signalling, where the enzyme may be tethered to the cytoplasmic tail of a cell surface receptor or a scaffold before catalysing reactions. In extracellular examples of such reactions the enzyme may be tethered to a scaffold before catalysing reactions.

Tethering can localise an enzyme near its substrate, and thus allow the production of regions in which the concentration of enzyme and substrate is much increased. As a consequence, tethering of enzymes may enhance reaction rates, improve reaction specificity, facilitate signal integration, or insulate signal networks within the same cell.

The investigation of tethered enzymatic catalysis is of interest in a number of research areas. It can provide “basic science” information regarding enzymatic activity, or can be used in industrial settings, such as drug screening, and investigation of drug mode of action.

Investigation of the kinetics of enzymatic reactions classically involves the determination of a single parameter the overall catalytic activity (k*_{cat}). This rate constant is equal to k_{cat}/K_{M}, where k_{cat} is the intrinsic catalytic rate and K_{M} is the Michaelis constant.

In tethered enzymatic reactions, the on-rate (kₒₙ) and off-rate (k_{off}) of localisation and the reach length (L), which provides an indication of the distance over which a tethered enzyme can catalyse reactions, is also considered, in addition to k*_{cat}. The reach length is determined by the mechanical properties of the tether and enzyme, including, but not limited to, the persistence length and the contour length of these components. The contour length of an object is its length when extended fully. For peptides, a good estimate of contour length can be obtained by multiplying the number of amino acid residues by 0.3 nm.

Surface plasmon resonance (SPR) is a technique used for biosensor applications. SPR involves the measurement of a resonance condition which is established when a frequency of incident photons matches a natural frequency of surface electrons. SPR measurements typically provide an output in terms of Resonance Units (RU) as a function of time, which may be termed as a sensorgram. In sensor applications a binding partner or ligand is immobilised on a surface of the sensor and another binding partner or analyte injected over the surface. A mass of material bound at the surface causes a change in RU over time.

The sensorgram may be used to determine an association rate (kₒₙ) and dissociation rate (k_{off}) of the binding partners. However it is difficult to determine the characteristics of more complex processes from the SPR data.

It is an object of embodiments of the invention to at least mitigate one or more of the problems of the prior art.

### BRIEF DESCRIPTION OF THE DRAWINGS

Embodiments of the invention will now be described by way of example only, with reference to the accompanying figures, in which:

FIG. 1 illustrates a tethered enzymatic surface plasmon resonance assay;

FIG. 2 is a schematic illustration of a system according to an embodiment of the invention;

FIG. 3 shows an illustration of SPR data;

FIG. 4 shows an illustration of a free phosphorylated peptide and a bound phosphorylated peptide separated by a distance;

FIG. 5 shows a method of determining characteristics of enzyme catalysis according to an embodiment of the invention;

FIG. 6 shows a comparison of parameters determined by an embodiment of the invention and SPR traces; and

FIG. 7 shows a variety of theoretical SPR data determined by an embodiment of the invention.

### DETAILED DESCRIPTION OF EMBODIMENTS OF THE INVENTION

In tethered enzymatic reactions, a binding domain associated with an enzyme attaches to a surface, thus serving to “tether” the enzyme. The tethered enzyme is able to interact with its substrate, and thereby catalyse the conversion of the substrate into the products of the enzymatic reaction. Enzymes that are not tethered, and thus remain in solution, may also cause catalysis of substrates.

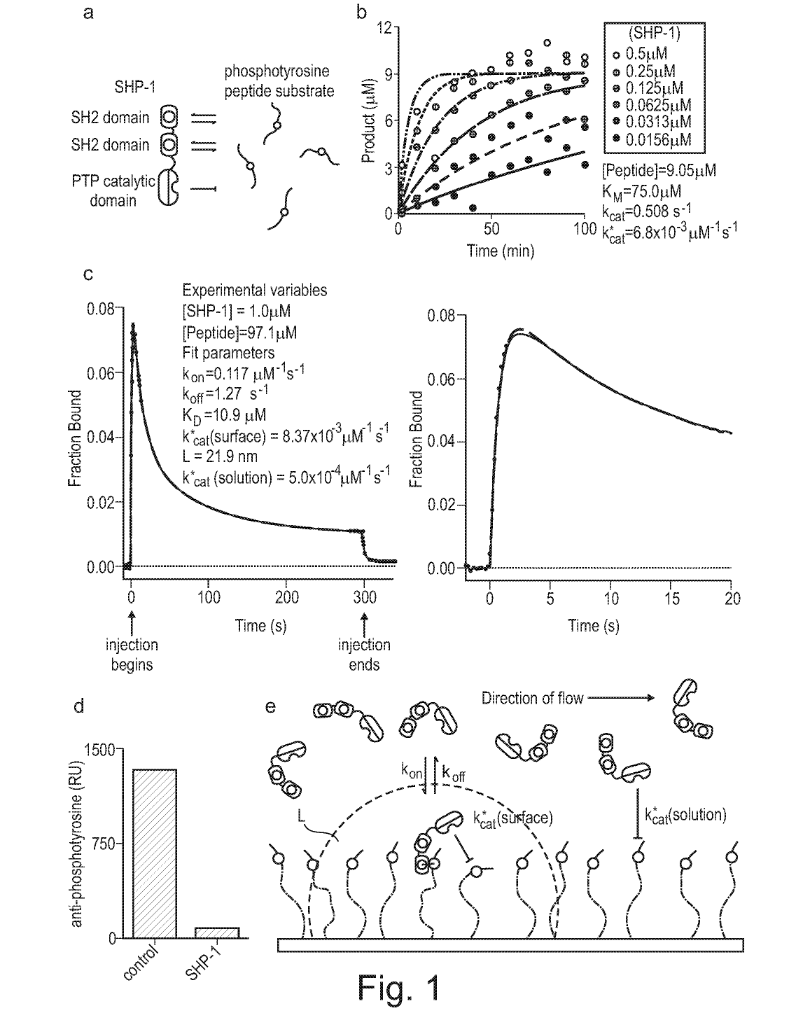

FIG. 1: A tethered enzymatic surface plasmon resonance assay for SHP-1. (a) Panel “a” shows a schematic of the domain structure of SHP-1 100, an enzyme comprising SH2 domains 110 that bind to phosphorylated tyrosine residues, thereby protecting them from de-phosphorylation, and a protein tyrosine phosphatase (PTP) domain 120. The PTP domain 120 catalyses de-phosphorylation of phosphorylated tyrosine residues.

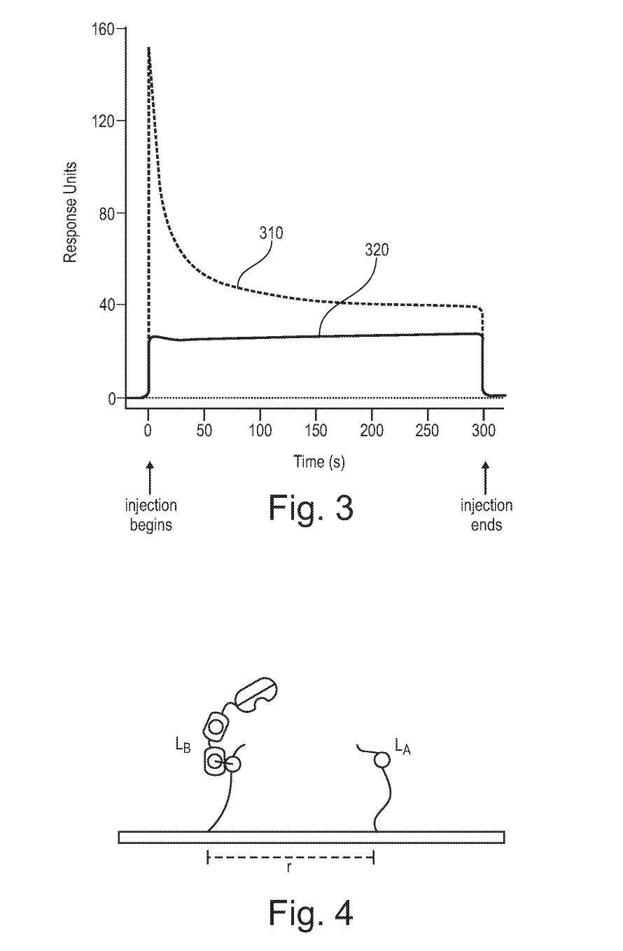

Panel “b” is a graph illustrating the results of a standard solution-based enzymatic assay showing the production of inorganic phosphate (the product of enzyme catalysed de-phosphorylation) over time for the indicated concentration of SHP-1 100 mixed with phosphorylated PEG12-ITIM substrate. The data are representative of two independent experiments. Progress curves are fit with a mathematical model to provide parameter estimates. Assays of this sort, known in the art, allow recovery of two parameters (k_{cat} and K_{M}) from which k*cat can be derived. In contrast methods of the invention allow not only determination of separate k*cat in respect of both enzyme in solution and enzyme tethered to the surface, but also determination of kₒₙ, k_{off}, and L (as defined elsewhere herein). Panel “c” presents a representative SPR trace for SHP-1 100 injected over a surface immobilised with a phosphorylated ITIM peptide derived from LAIR-1 on a 28 repeat polyethylene glycol linker (PEG28-ITIM). A fit of a multi-centre particle distribution (MPD) model according to an embodiment of the invention as will be described provides estimates for the indicated parameters kₒₙ, k_{off}, k_{D}, k*_{cat} for surface bound enzyme, k*cat for enzyme in solution, and L (reach parameter). Unprocessed SPR data is shown in FIG. 3.

Panel “d” is a bar chart illustrating the degree of anti-phosphotyrosine antibody binding that occurs when the antibody is injected at the end of the experiment. As can be seen, antibody binding is reduced in the experimental flow cell (designated “SHP-1”) compared to a buffer-injected flow cell with equivalent peptide levels (designated “control”) illustrating the decrease in PEG28-ITIM phosphorylation occurring as a result of SHP-1 enzyme activity.

Panel “e” is a schematic diagram of reactions taking place when SHP-1 100 is injected over a surface 130 immobilised with phosphorylated peptides 140. It can be seen that SH2 domains 110 of SHP-1 100 interact with phosphorylated residues 150 on the phosphorylated peptides 140, thereby tethering the enzyme 100 to the surface 130. This interaction is controlled by the association and dissociation constants (kₒₙ and k_{off} respectively). The shaded area 160 illustrates the reach range (L) over which a tethered enzyme 100 can catalyse de-phosphorylation of its substrate 140. Note that peptide anchoring is displayed in 1D for clarity but because the surface consists of a dextran matrix onto which peptides randomly couple they are anchored in 3D. On the right hand side of the panel it is illustrated that un-tethered enzymes in solution are also able to catalyse de-phosphorylation of the bound phosphorylated substrate peptides 140. Catalytic activity can be deduced in respect of both the tethered (k*_{cat}(surface)) and untethered (k*_{cat}(solution)) SHP-1 enzyme 100.

Further details regarding the enzyme catalysis suitable for analysis by the method and apparatus of the invention are defined below.

Suitably, any enzyme-substrate interaction may be analysed by the invention.

In a suitable embodiment, the enzyme is an intracellular enzyme. Suitably, the enzyme is one associated with intracellular signalling.

Suitably, the enzyme is an activating enzyme or a de-activating enzyme. Suitably therefore the enzyme may catalyse an activation reaction or a de-activation reaction.

Suitably, the invention may determine characteristics of an activation reaction or a deactivation reaction. Suitably, an enzymatic activation reaction or an enzymatic deactivation reaction.

Suitably, the enzyme may modify the substrate. Suitably the enzyme may catalyse activation or de-activation reactions by modification of the substrate. Suitably the enzyme may modify the substrate such that binding of the substrate can occur.

Suitably, the enzyme may catalyse post-translational modification, suitably of a substrate. Suitably, the invention may determine characteristics of a post-translational modification reaction.

Suitably the enzyme may modify the substrate by phosphorylation or dephosphorylation, ubiquitination or de-ubiquitination, alkylation or de-alkylation, acylation or de-acylation, amidation or de-amidation, glycosylation or de-glycosylation, hydroxylation or de-hydroxylation and the like.

Suitably, the enzyme may catalyse a phosphorylation reaction or a de-phosphorylation reaction, an ubiquitination or de-ubiquitination reaction, alkylation or de-alkylation, acylation or de-acylation, amidation or de-amidation, glycosylation or de-glycosylation, hydroxylation or de-hydroxylation and the like.

In one embodiment, the enzyme may catalyse a phosphorylation reaction or a de-phosphorylation reaction. Suitably therefore, the enzyme is a phosphatase or a kinase.

Suitably, in one embodiment, the invention may determine characteristics of a phosphorylation reaction or a dephosphorylation reaction. Suitably, an enzymatic phosphorylation reaction or an enzymatic dephosphorylation reaction.

Suitably, the enzyme is a tyrosine phosphatase, or a tyrosine kinase. Suitably, any class of tyrosine phosphatase, or tyrosine kinase enzyme.

Suitably, the invention may determine characteristics of tyrosine phosphatase catalysis or tyrosine kinase catalysis.

In one embodiment, the enzyme is a phosphatase enzyme selected from SHP-1, SHP-2, CD45 or CD148. In one embodiment, the invention may determine characteristics of SHP-1, SHP-2, CD45 or CD148 catalysis.

In one embodiment, the enzyme is SHP-1.

In one embodiment, the enzyme is a kinase enzyme selected from an SRC family kinase, or a SYK family kinase. In one embodiment, the enzyme is a SRC family kinase selected from Lck, Fyn, or Src. In one embodiment, the enzyme is a SYK family kinase selected from Syk and ZAP-70. In one embodiment, the invention may determine characteristics of Lck, Fyn, Src, Syk or ZAP-70 catalysis.

Suitably, the enzyme may be tethered or non-tethered. Suitably, the enzyme may be in solution. Suitably the substrate may be tethered or non-tethered. Suitably, the substrate may be in solution.

Suitably, the invention may determine characteristics of tethered enzyme catalysis or enzyme catalysis in solution.

In one embodiment, the invention may determine characteristics of a tethered enzymatic phosphorylation reaction, suitably a tethered kinase reaction, or a tethered enzymatic dephosphorylation reaction, suitably a tethered phosphatase reaction. In a suitable embodiment the enzyme is a potential target for therapeutic manipulation. The enzyme may be a drug target, and the methods of the invention applied as part of a drug screening study. Suitably the methods of the invention may be used to investigate the method of action of known drugs, or putative drugs, on enzyme targets.

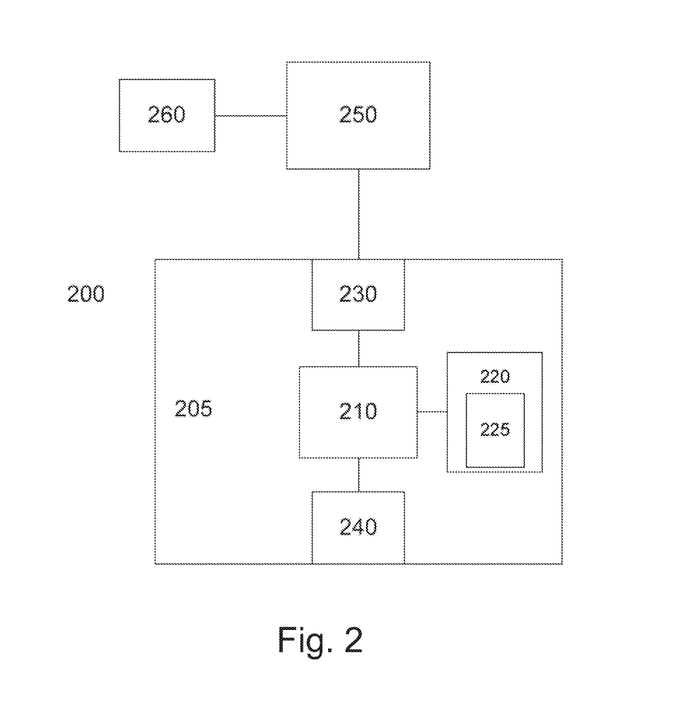

FIG. 2 illustrates a biosensor system 200 according to an embodiment of the invention. The system comprises an apparatus 205 which may be a computing unit 205 and a surface plasmon resonance (SPR) instrument 250 associated with a biosensor 260.

The biosensor 260 comprises a biosensor surface which may be a metallic electrically-conducting surface, such as gold, although other materials may be used. The surface may be covered with a binding material or ligand, which may be dextran or a dextran matrix, although other materials may be used. The biosensor 260 may be a sensor chip, which is disposable and may be used for only one measurement. The biosensor 260 may be located within, or part of, a flow cell which allows a flow of liquid past the biosensor 260. In some embodiments a control flow cell and control biosensor may be provided which is subject to a flow of liquid which does not comprise an analyte to provide control SPR data.

The SPR instrument 250 is an apparatus for measuring a resonance angle of the biosensor 260, in particular of the biosensor 260 surface, as will be appreciated. The SPR instrument 250 is arranged in use to direct polarised light toward the surface. The instrument 250 determines an angle of minimum intensity reflected light. The angle of minimum intensity changes as molecules bind to and dissociate from the surface. The SPR instrument 250 stores SPR data indicative of the minimum intensity angle over time. The SPR instrument 250 may be a BIAcore (R) instrument from GE Healthcare, although it will be appreciated that other SPR instruments may be used. Such BIAcore instruments provide SPR data in the form of Resonance Units (RU). 1 RU approximately relates to 1 picogram of material per square millimetre of the biosensor surface, although it will be appreciated that SPR data in other formats or units may be utilised. For the purpose of illustration RU will be referred to herein although it will be realised that this is merely an example.

The SPR instrument is communicably coupled to the computing unit 205. The communicable coupling may include one or more computer networks, such as including the Internet. The SPR instrument 250 is arranged to output SPR data to the computing unit 205. The computing unit 205 is an apparatus which is arranged to determine characteristics of enzyme catalysis based on the received SPR data. The computing unit 205 is arranged to determine one or more characteristics of the enzyme catalysis comprising one or more of catalytic rate (k*_{cat}), association (k₀) and dissociation (k_{off}) of an enzyme. In some embodiments the computing unit 205 may further determine a reach length L.

The computing unit 205 comprises a processing unit 210. The processing unit may comprise one or more processors for operatively executing computer program instructions in the form of computer software. The one or more processors may be electronic processing devices. The computer software implements a method according to an embodiment of the invention as will be explained. In other embodiments, the processing unit may be a unit configured to perform a method according to an embodiment of the invention to determine the one or more characteristics.

The computing unit 205 comprises a memory unit 220 for storing data therein. The memory unit 220 may be formed by one or more memory devices. The computer software may be stored in the memory unit 220 for execution by the processing unit 210. The computing unit 205 comprises one or both of an interface 230 for receiving the SPR data from the SPR instrument 250 and an output unit 240 for outputting an indication of the one or more characteristics. The interface 230 may for receiving the SPR data may comprise an electrical input for receiving an electrical signal indicative of the SPR data.

The memory unit 220 stores data indicative of a multi-centre particle density (MPD) model 225. The MPD model 225 comprises a multi-centre particle distribution system of coupled partial differential equations (PDEs), as will be explained. Thus the MPD model 225 may be considered as a hybrid MPD-PDE model. Such models have been used in the field of solid state physics. However the present inventors have found that such models, unexpectedly, may be used to model SPR data, in particular SPR data indicative of characteristics of enzyme catalysis. The MPD model 225 is used by the processing unit 210 to determine the one or more characteristics of the enzyme catalysis, data relating to which is provided by the SPR instrument 250. In use, the memory unit 220 may store the received SPR data as will be explained.

The MPD model 225 offers advantages over stochastic simulation techniques, which may be considered impractical due to their long computation times. Furthermore, the deterministic MPD model 225 offers advantages over models based on standard partial-differential-equations which have been found not to fit the SPR data adequately.

FIG. 3 illustrates an example of SPR data output by the SPR instrument 250 and received by the computing unit 205. The SPR data may be as stored in the memory unit 220. Illustrated in FIG. 3 is a first SPR trace 310 for an experimental flow cell and a second SPR trace 320 for a control reference flow cell. The analyte is injected over both flow cells but only the experimental flow cell contains the immobilised substrate. The first SPR trace 310 shows a change in RU of the biosensor 260 over time due to binding to the surface of the biosensor 260. The computing unit 205 may be arranged to pre-process the SPR data by subtracting the second SPR trace 320 from the first SPR trace 310. The second SPR trace 320 may be used as reference SPR data for a plurality of first SPR traces 310 i.e. control SPR data need not be produced for every experimental run of the biosensor 260, although it may be useful in some embodiments to do so. Furthermore, the pre-processing may comprise normalising the first SPR trace 310, or the resulting subtracted SPR data, to a maximum theoretical binding value.

In some embodiments of the invention, a conversion factor between mass on the biosensor 260 surface and RU is determined. The conversion factor may be used to determine a concentration of peptide. The conversion factor is determined by injecting a plurality, such as four, concentrations of SHP-1 over a control flow cell comprising the biosensor 260 and measuring the raw RU change. The raw RU change is related to differences in buffer composition and the mass of SHP-1 in the evanescent field above the surface (˜100-200 nm), which constitutes the volume that is observable to the SPR instrument 250. The raw RU may then be plotted or determined over different concentrations of injected SHP-1 which produced straight lines whose slope has units of RU per g/L of SHP-1. The y-intercept of these plots is related to small differences in buffer composition between the sample and running buffer, which were negligible and irrelevant for determining the conversion factor. A plurality, such as seven, independent slopes were calculated and averaged to give a conversion factor between RU and g/L of protein at the biosensor 260 surface: 149±15 RU per g/L (±SEM). This conversion factor, together with the molecular weight of the peptide, was used to convert between the RU of peptide immobilised and the molar concentration at the surface of the biosensor 260.

In embodiments of the invention, a component of the MPD model 225 is a calculation of a local substrate concentration that a tethered enzyme experiences. Referring to FIG. 4, it may be assumed that the motion of a free phosphorylated peptide (state A) and the motion of SHP-1 bound to a phosphorylated peptide (state B) can both be approximated by the worm-like-chain model, which is used as a polymer model. This model provides the probability of finding the tip of the polymer at position (or distance) r as defined by Equation 1 in the Equations Appendix, where l=\sqrt{l_{c}lₚ} where l_{c} is the contour length and lₚ is the persistence length. The contour length is the length of an object from ‘tip to toe’ when you stretch it fully. For peptides, an estimate may be the number of amino acids multiplied by 0.3 nm/amino acids (where 0.3 nm is the c-alpha to c-alpha distance). When applied to the free phosphorylated substrate this probability is taken to be the position of the phosphorylated tyrosine residue with l=L_{A}. When applied to bound SHP-1 this probability is taken to be the position of the catalytic pocket of the phosphatase domain with l=L_{B}.

Using these probabilities, the concentration of substrate, σ(r), that a tethered enzyme will experience when they are anchored a distance of r apart, as in FIG. 4, can be calculated as in Equation 2 in the Equations Appendix, where the integration is over all space. Without loss of generality, letting r=r\hat{z}, which leads to Equation 3 in the Appendix.

Rearranging the equation and including the variable substitution q=(r′)²+r²−2rr′ cos(θ) leads to Equation 4 in the Equations Appendix, where L=\sqrt{(L_{A}²+L_{B}²)}.

As explained, the MPD model 225 comprises a multi-centre particle distribution system of coupled partial differential equations. The MPD model 225 can be defined as ρ_{m,m}, as defined by Equation 5 in the Equations Appendix, where bold-face denotes vector quantities. Equation 5 relates to a tethered-dephosphorylation reaction, whereas Equation 5′ relates to a tethered-phosphorylation reactions. The convention of using to relate to tethered-phosphorylation reactions applies through the equations. In Equation 5/5′ m relates to A particles, m′ relates to B particles and m″ relates to C particles.

The explicit expression for the first 5 MPDs may be given by the equations listed in Equation 6 in the Equations Appendix, where n_{A} and n_{B} are defined as the concentration of A and B, respectively, and X_{A}, X_{B} and Y are defined as the autocorrelation function for A, the autocorrelation function for B, and the pair correlation function between A and B, respectively.

The general set of PDEs governing the dynamics of the MPDs based on the reactions outlined above can be defined by Equation 7 in the Equations Appendix.

The explicit expression for the first 5 MPD-PDEs can be defined by the equations listed in Equation 8 in the Equations Appendix.

Equation 9 gives Kirkwood's approximation, which can be used to uncouple the infinite hierarchy of the PDEs listed in Equation 8. This leads to definitions for ρ_{1,2} and ρ_{2,1}, as given by Equations 10 and 11 in the Equations Appendix. For a tethered-phosphorylation reaction, Equations 9-11 may be replaced with a single general equation, namely Equation 9′.

The derivatives of the first 5 MPDs can be expressed in terms of their definitions (n_{A}, n_{B}, X_{A}, X_{B}, and Y) to give Equation 12 in the Equations Appendix.

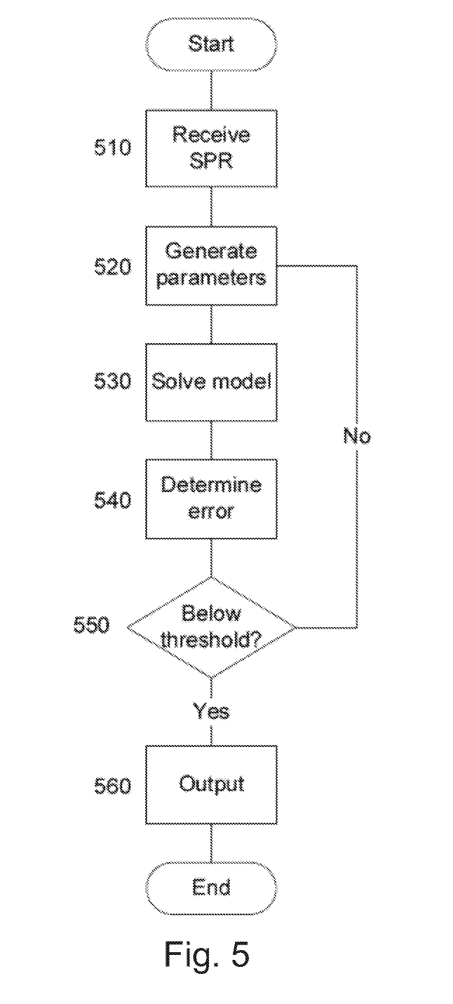

A method 500 of determining characteristics of enzyme catalysis according to an embodiment of the invention is illustrated in FIG. 5.

The method 500 comprises a step 510 of receiving SPR data. The SPR data is received from SPR instrument 250. The SPR data is indicative of binding of the enzyme and a substrate. The SPR data may comprise an SPR trace such as the first SPR trace 310 illustrated in FIG. 3. The SPR data represents a change in resonance angle of the biosensor 260 over a period of time. The SPR data may be in units of RU. The SPR data is received at the computing unit 205 from the SPR instrument 250. The SPR data does not have to be received directly from the SPR instrument at the computing unit 205 and may be received over one or more communications networks including the internet. The SPR data may be received via interface 230 stored in the memory unit 220 of the computing unit 205. Step 510 may, in some embodiments, comprise pre-pre-processing the SPR data, for example to remove control SPR data such as the second SPR trace 320 and normalising the SPR data to a reference value.

Steps 520-550 comprise using the MPD model 225 to estimate the one or more parameters, as will be explained. The estimate of the one or more parameters determined is compared against the SPR data and steps iteratively repeated until the estimate meets one or more conditions.

Step 520 of the method comprises, in a first iteration of steps 520-550, determining parameters for the MPD model 225. Using the derivatives obtained before in Equation 12 along with the simplified expressions for ρ_{1,2} and ρ_{2,1} obtained using Kirkwood's approximation, the PDEs for the first 5 MPDs comprising the MPD model 225 can be simplified in the form of Equation 13 in the Equations Appendix. The parameters for the MPD model 225 may be initial conditions for the MPD model 225. The initial conditions may comprise one or more of: η_{A}(t=0), η_{B}(t=0), X_{A}(t=0, r), X_{B} (t=0, r) and Y(t=0, r). Whilst other initial values may be used, example initial values may be: η_{A}(t=0)=1, η_{B}(t=0)=0, X_{A}(t=0,r)=1, X_{B} (t=0, r)=1 and Y(t=0, r)=1.

The numerical solution of the integral MPD-PDE system given by Equation 13 can be obtained by noting there are two distinct types of integrals. The first integral may appear in the equation for η_{A}, and can be evaluated using |r′|=r′ to obtain the Equation 14 in the Equations Appendix.

Similarly, the second integral may appear in the equations for Y and X_{A}, and can be evaluated using q=|r−r′|=\sqrt{r²+(r′)²−2rr′ cos(θ)} where, without loss of generality, it is assumed that r=r\hat{z} thus leading to Equation 15 in the Equations Appendix.

Using the definitions given by Equations 14 and 15, the definition of σ(r) shown in Equation 4, and by rescaling η_{A} and η_{B} by [Peptide] and r by L, the non-dimensional MPD-PDE system shown by Equation 16 can be derived. Whilst other initial values may be used, example initial values may be: η_{A}(t=0)=1, η_{B}(t=0)=0, X_{A}(t=0, r)=1, X_{B} (t=0, r)=1 and Y(t=0, r)=1.

Step 520 may also comprise, in the first iteration, determining values for one or both of numerical parameters ΔR for spatial discretisation and Rₘₐₓ for an integration upper bound. Example values may be ΔR=0.05 and Rₘₐₓ=4.5. It has been found that these example values introduce errors substantially smaller than experimental noise whilst maintaining computation efficiency for the method 500. In some embodiments these numerical parameters may be set as default values.

Step 520 further comprises determining values for the one or more fitting parameters, which may comprise p₁, p₂, p₃, p₄ and p₅. The values for the fitting parameters may be randomly selected in each iteration of step 530. The 5 fitting parameters (p₁, p₂, p₃, p₄ and p₅,) may be related to the 5 biophysical constants. The parameters may be of the form p₁=kₒₙ [SHP-1], p₂=k_{off}, p₃=k_{cat}* (surface)×[Peptide], p₄=k_{cat}*, (surface)\L³, and p₅=k_{cat}* (solution)×[SHP-1].

Concentrations of an enzyme (such as SHP-1) and its peptide substrate can be set to desired levels, for example as part of a standard calibration. Alternatively, the concentrations of these agents can be readily determined by means well known to those skilled in the art.

Markov-Chain Monte-Carlo (MCMC) analysis of the experimental SPR data in FIG. 1c has shown that all 5 biophysical parameters can be determined independently of each other.

In step 530 the MPD model 225 is solved based on the fitting parameters determined in step 520.

In step 540 an error between the MPD model 225 and the SPR data received in step 510 is determined. The error may be determined as a sum-of-squares error.

In step 550 it is determined whether the error determined in step 540 is less than a predetermined error threshold. If the error is less than the error threshold, then the method moves to step 550. If, however, the error is greater than the threshold the method returns to step 520.

If step 520 is repeated new values for at least some of the parameters determined in a previous iteration of step 520 are determined. Specifically, in some embodiments, new values for the fitting parameters comprise p₁, p₂, p₃, p₄ and p₅ may be determined.

In some embodiments, steps 520-550 may be performed by Matlab function lsqcurvefit_which is provided with the SPR data as an input and the MPD model 225 and then determines the fitting parameters.

The fitting parameters or one or more of the biophysical constants are output by the computing unit in step 560. The fitting parameters or biophysical constants may be output by the computing unit via the output unit 240 as the indication of the one or more characteristics of the enzyme catalysis. The output unit 240 may be an interface unit to, for example a data communications network which outputs data indicative of the fitting parameters onto the communications network for reception by another device. In this case, the output unit 240 is arranged for outputting an electrical signal indicative of the fitting parameters. However in other embodiments the output unit 240 may comprise a user interface, such as a visual display unit (VDU) for visually outputting the fitting parameters to a user, such as by display on the VDU. In particular, in step 560 an indication of one or more of catalytic rate (k_{cat}*), association (kₒₙ) and dissociation (k_{off}) of an enzyme. In some embodiments the computing unit 205 may further output the reach length L

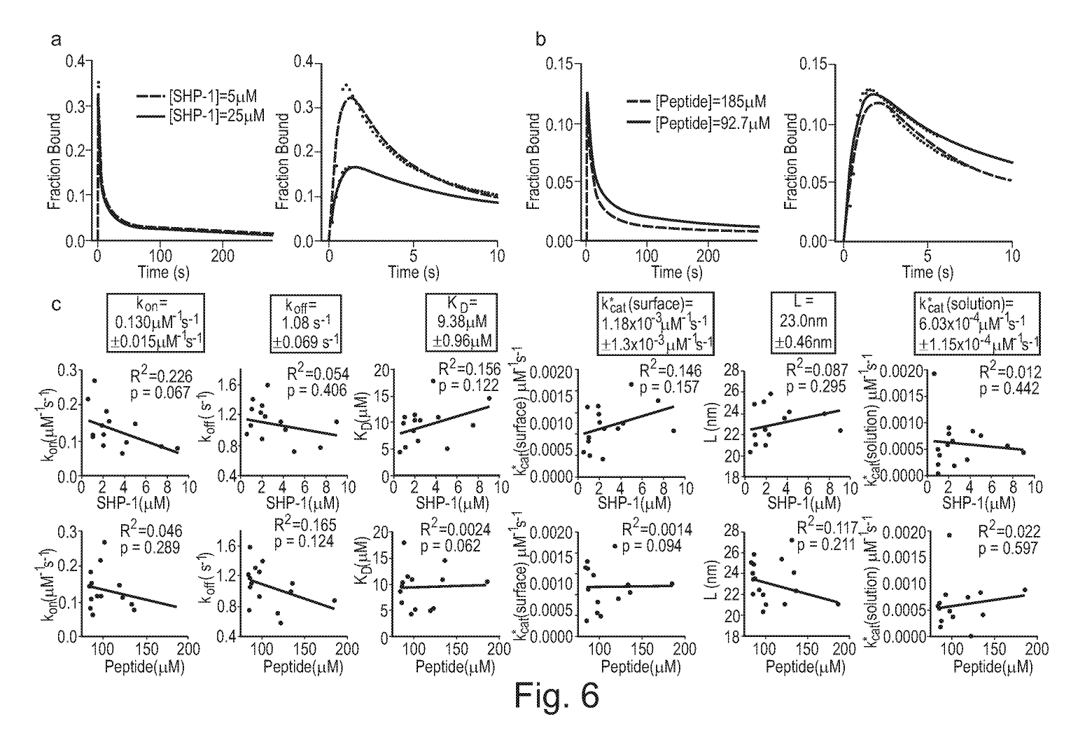

Referring to FIG. 6, fitted parameters are shown independent of experimental variables. Representative SPR traces (black) and MPD model 225 fits (colour) are shown for (a) two SHP-1 concentrations and (b) two initial peptide concentrations. (c) Plots of fitted parameters versus SHP-1 concentration (upper row) and peptide concentration (lower row) with linear regression fits (R and p values are indicated). Averages of fit parameters with SEMs from all experiments are shown in boxes (n=15). All experiments are performed using wild-type SHP-1 and phosphorylated PEG28-ITIM peptides.

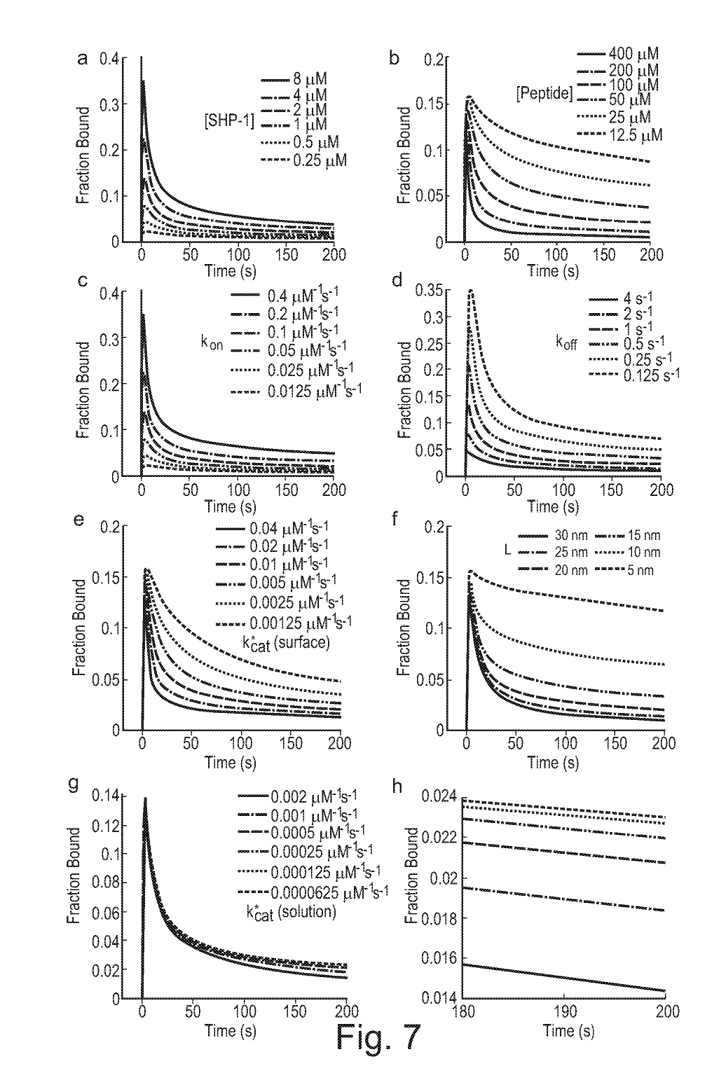

FIG. 7 shows theoretical SPR traces generated by the MPD model 225 according to an embodiment of the invention. Each panel depicts the fraction of SHP1 bound over time when varying the (a) SHP-1 concentration, (b) peptide concentration, (c) kₒₙ, (d) k_{off} (e) k*_{cat} (surface), (f) L, and (g) k*_{cat} (solution). (h) An expanded view of the k_{cat} (solution) curves at late time points is also shown to clarify subtle difference between curves. Note that variation in peptide concentration changes the shape of the curve as a result of a different fraction of peptides being surface versus solution dephosphorylated.

Default parameter values are [SHP-1]=2 μM, [peptide]=100 μM, kₒₙ=0.1 μM⁻¹ s⁻¹, koff=1 s⁻¹, k_{cat}* (surface)=0.01 μM⁻¹ s⁻¹, L=20 nm, and k_{cat}* (solution)=0.0005⁻¹ s⁻¹.

It will be appreciated that embodiments of the present invention can be realised in the form of hardware, software or a combination of hardware and software. Any such software may be stored in the form of volatile or non-volatile storage such as, for example, a storage device like a ROM, whether erasable or rewritable or not, or in the form of memory such as, for example, RAM, memory chips, device or integrated circuits or on an optically or magnetically readable medium such as, for example, a CD, DVD, magnetic disk or magnetic tape. It will be appreciated that the storage devices and storage media are embodiments of machine-readable storage that are suitable for storing a program or programs that, when executed, implement embodiments of the present invention. Accordingly, embodiments provide a program comprising code for implementing a system or method as claimed in any preceding claim and a machine readable storage storing such a program. Still further, embodiments of the present invention may be conveyed electronically via any medium such as a communication signal carried over a wired or wireless connection and embodiments suitably encompass the same.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed. The claims should not be construed to cover merely the foregoing embodiments, but also any embodiments which fall within the scope of the claims.

Equations

$$\begin{matrix} P ( r ) = {( \frac{3}{2 π {l}^{2}} )}^{3 / 2} exp ( - \frac{3 r \cdot r}{2 {l}^{2}} ) & Equation 1 \\ σ ( r ) = \int {P}_{A} ( {r}^{′} ) {P}_{B} ( {r}^{′} - r ) {d}^{3} {r}^{′} & Equation 2 \\ σ ( r ) = {( \frac{3}{2 π {L}_{A}^{2}} )}^{3 / 2} {( \frac{3}{2 π {L}_{B}^{2}} )}^{3 / 2} {\int }_{0}^{2 π} d φ {\int }_{0}^{π} d θ {\int }_{0}^{∞} d {{r}^{′} ( {r}^{′} )}^{2} sin ( θ ) \times exp ( - \frac{3 {( {r}^{′} )}^{2}}{2 {L}_{A}^{2}} ) exp ( - \frac{3}{2 {L}_{B}^{2}} ( {( {r}^{′} )}^{2} + {( r )}^{2} - 2 {rr}^{′} cos ( θ ) ) ) & Equation 3 \\ σ ( r ) = {( \frac{3}{2 π {L}^{2}} )}^{3 / 2} exp ( - \frac{3 {r}^{2}}{2 {L}^{2}} ) & Equation 4 \\ {ρ}_{m , {m}^{′}} = 〈 {\prod }_{i = 1}^{m} {n}_{A} ( {r}_{i} , t ) {\prod }_{j = 1}^{{m}^{′}} {n}_{B} ( {r}_{j}^{′} , t ) 〉 & Equation 5 \\ {ρ}_{m , {m}^{′} , {m}^{″}} = 〈 {\prod }_{i = 1}^{m} {n}_{A} ( {r}_{i} , t ) {\prod }_{j = 1}^{{m}^{′}} {n}_{B} ( {r}_{j}^{′} , t ) {\prod }_{k = 1}^{{m}^{″}} {n}_{C} ( {r}_{k}^{″} , t ) 〉 & Equation 5 ’ \\ {ρ}_{1 , 0} = 〈 {n}_{A} ( {r}_{1} , t ) 〉 ≡ {n}_{A} ( t ) \\ {ρ}_{0 , 1} = 〈 {n}_{B} ( {r}_{1}^{′} , t ) 〉 ≡ {n}_{B} ( t ) \\ {ρ}_{2 , 0} = 〈 {n}_{A} ( {r}_{1} , t ) {n}_{A} ( {r}_{2} , t ) 〉 ≡ {n}_{A}^{2} ( t ) {X}_{A} ( t , {r}_{1} - {r}_{2} ) \\ {ρ}_{0 , 2} = 〈 {n}_{B} ( {r}_{1}^{′} , t ) {n}_{B} ( {r}_{2}^{′} , t ) 〉 ≡ {n}_{B}^{2} ( t ) {X}_{B} ( t , {r}_{1}^{′} - {r}_{2}^{′} ) \\ {ρ}_{1 , 1} = 〈 {n}_{A} ( {r}_{1} , t ) {n}_{B} ( {r}_{1}^{′} , t ) 〉 ≡ {n}_{A} ( t ) {n}_{B} ( t ) Y ( t , {r}_{1} - {r}_{1}^{′} ) & Equation 6 \\ {ρ}_{2 , 0 , 0} = 〈 {n}_{A} ( {r}_{1} , t ) {n}_{A} ( {r}_{2} , t ) 〉 ≡ {n}_{A}^{2} ( t ) {X}_{A} ( {r}_{1} - {r}_{2} , t ) \\ {ρ}_{0 , 2 , 0} = 〈 {n}_{B} ( {r}_{1}^{′} , t ) {n}_{B} ( {r}_{2}^{′} , t ) 〉 ≡ {n}_{B}^{2} ( t ) {X}_{B} ( {r}_{1}^{′} - {r}_{2}^{′} , t ) \\ {ρ}_{0 , 0 , 2} = 〈 {n}_{C} ( {r}_{1}^{″} , t ) {n}_{C} ( {r}_{2}^{″} , t ) 〉 ≡ {n}_{C}^{2} ( t ) {X}_{C} ( {r}_{1}^{″} - {r}_{2}^{″} , t ) \\ {ρ}_{1 , 1 , 0} = 〈 {n}_{A} ( {r}_{1} , t ) {n}_{B} ( {r}_{1}^{′} , t ) 〉 ≡ {n}_{A} ( t ) {n}_{B} ( t ) {Y}_{AB} ( {r}_{1} - {r}_{1}^{′} , t ) \\ {ρ}_{0 , 1 , 1} = 〈 {n}_{B} ( {r}_{1}^{′} , t ) {n}_{C} ( {r}_{1}^{″} , t ) 〉 ≡ {n}_{B} ( t ) {n}_{C} ( t ) {Y}_{BC} ( {r}_{1}^{′} - {r}_{1}^{″} , t ) \\ {ρ}_{1 , 0 , 1} = 〈 {n}_{A} ( {r}_{1} , t ) {n}_{C} ( {r}_{1}^{″} , t ) 〉 ≡ {n}_{A} ( t ) {n}_{C} ( t ) {Y}_{A C} ( {r}_{1}^{″} - {r}_{1} , t ) , & Equation 6 ’ \\ \frac{\partial {ρ}_{m , {m}^{′}}}{\partial t} = - {\sum }_{i = 1}^{m} {\sum }_{j = 1}^{{m}^{′}} μ σ ( {r}_{i} - {r}_{j}^{′} ) {ρ}_{m , {m}^{′}} - {\sum }_{i = 1}^{m} \int μσ ( {r}_{i} - {r}_{{m}^{′} + 1}^{′} ) {ρ}_{m , {m}^{′} + 1} {d}^{3} {r}_{{m}^{′} + 1}^{′} + {k}_{o n}^{*} ( {m}^{′} {ρ}_{m + 1 , {m}^{′} - 1} ) + {k}_{off} ( m {ρ}_{m - 1 , {m}^{′} + 1} ) - ( λ + {k}_{o n}^{*} ) ( m {ρ}_{m , {m}^{′}} ) - {k}_{off} ( {m}^{′} {ρ}_{m , {m}^{′}} ) & Equation 7 \\ \frac{\partial {ρ}_{m , {m}^{′} , {m}^{″ }}}{\partial t} = {mk}_{off} {ρ}_{m - 1 , {m}^{′} + 1 , {m}^{″}} + {m}^{′} {k}_{on}^{*} {ρ}_{m + 1 , {m}^{′} - 1 , {m}^{″}} - {mk}_{o n}^{*} {ρ}_{m , {m}^{′} , {m}^{″}} - {m}^{′} {k}_{off} {ρ}_{m , {m}^{′} , {m}^{″}} + m λ {ρ}_{m - 1 , {m}^{′} , {m}^{″} + 1} - {m}^{″} λ {ρ}_{m , {m}^{′} , {m}^{″}} - {\sum }_{i = 1}^{{m}^{′}} {\sum }_{j = 1}^{{m}^{″}} {k}_{cat}^{*} σ ( {r}_{i}^{′} - {r}_{j}^{″} ) {ρ}_{m , {m}^{′} , {m}^{″}} - & Equation 7 ’ \\ {\sum }_{i = 1}^{{m}^{″}} \int {k}_{cat}^{*} σ ( {r}_{{m}^{′} + 1}^{′} - {r}_{i}^{″} ) {ρ}_{m , {m}^{′} + 1 , {m}^{″}} {d}^{d} {r}_{{m}^{′} + 1}^{′} + {\sum }_{i = 1}^{m} {\sum }_{j = 1}^{{m}^{′}} {k}_{cat}^{*} σ ( {r}_{j}^{′} - {r}_{i} ) {ρ}_{m - 1 , {m}^{′} , {m}^{″} + 1} + {\sum }_{i = 1}^{m} \int {k}_{cat}^{*} σ ( {r}_{{m}^{′} + 1}^{′} - {r}_{i} ) {ρ}_{m - 1 , {m}^{′} + {m}^{″} + 1} {d}^{d} {r}_{{m}^{′} + 1}^{′} & \\ \frac{\partial {ρ}_{1 , 0}}{\partial t} = - ( {k}_{on}^{*} + λ ) {ρ}_{1 , 0} + {k}_{off} {ρ}_{0 , 1} - \int μ σ ( {r}_{1} - {r}_{1}^{′} ) {ρ}_{1 , 1} {d}^{3} {r}_{1}^{′} \\ \frac{\partial {ρ}_{0 , 1}}{\partial t} = {k}_{on}^{*} {ρ}_{1 , 0} - {k}_{off} {ρ}_{0 , 1} \\ \frac{\partial {ρ}_{1 , 1}}{\partial t} = - ( {k}_{on}^{*} + {k}_{off} + λ ) {ρ}_{1 , 1} + {k}_{on}^{*} {ρ}_{2 , 0} + {k}_{off} {ρ}_{2 , 0} + \\ μ σ ( {r}_{1} - {r}_{1}^{′} ) {ρ}_{1 , 1} - \int μ σ ( {r}_{1} - {r}_{2}^{′} ) {ρ}_{1 , 2} {d}^{3} {r}_{2}^{′} & Equation 8 \\ \frac{\partial {ρ}_{2 , 0}}{\partial t} = 2 {k}_{off} {ρ}_{1 , 1} - 2 ( {k}_{on}^{*} + λ ) {ρ}_{2 , 0} - \\ \int μ σ ( {r}_{1} - {r}_{1}^{′} ) {ρ}_{2 , 1} {d}^{3} {r}_{1}^{′} - \int μσ ( {r}_{2} - {r}_{1}^{′} ) {ρ}_{2 , 1} {d}^{3} {r}_{1}^{′} \\ \frac{\partial {ρ}_{0 , 2}}{\partial t} = 2 {k}_{on}^{*} {ρ}_{1 , 1} - 2 {k}_{off} {ρ}_{0 , 2} & \\ 〈 n ( {r}_{1} ) n ( {r}_{2} ) n ( {r}_{3} ) 〉 \approx \frac{〈 n ( {r}_{1} ) n ( {r}_{2} ) 〉 〈 n ( {r}_{1} ) n ( {r}_{3} ) 〉 〈 n ( {r}_{2} ) n ( {r}_{3} ) 〉}{〈 n ( {r}_{1} ) 〉 〈 n ( {r}_{2} ) 〉 〈 n ( {r}_{3} ) 〉} , & Equation 9 \\ 〈 n ( {r}_{1} ) n ( {r}_{2} ) n ( {r}_{3} ) 〉 \approx \frac{〈 n ( {r}_{1} ) n ( {r}_{2} ) 〉 〈 n ( {r}_{2} ) n ( {r}_{3} ) 〉 〈 n ( {r}_{3} ) n ( {r}_{1} ) 〉}{〈 n ( {r}_{1} ) 〉 〈 n ( {r}_{2} ) 〉 〈 n ( {r}_{3} ) 〉} & Equation 9 ’ \\ {ρ}_{1 , 2} \approx \frac{〈 {n}_{A} ( {r}_{1} ) {n}_{B} ( {r}_{1}^{′} ) 〉 〈 {n}_{A} ( {r}_{1} ) {n}_{B} ( {r}_{2}^{′} ) 〉 〈 {n}_{B} ( {r}_{1}^{′} ) {n}_{B} ( {r}_{2}^{′} ) 〉}{〈 {n}_{A} ( {r}_{1} ) 〉 〈 {n}_{B} ( {r}_{1}^{′} ) 〉 〈 {n}_{B} ( {r}_{2}^{′} ) 〉} = {n}_{A} {n}_{B}^{2} {X}_{B} ( {r}_{1}^{′} - {r}_{2}^{′} ) Y ( {r}_{1} - {r}_{1}^{′} ) Y ( {r}_{1} - {r}_{2}^{′} ) & Equation 10 \\ {ρ}_{2 , 1} \approx \frac{〈 {n}_{A} ( {r}_{1} ) {n}_{A} ( {r}_{2} ) 〉 〈 {n}_{A} ( {r}_{1} ) {n}_{B} ( {r}_{1}^{′} ) 〉 〈 {n}_{A} ( {r}_{2} ) {n}_{B} ( {r}_{1}^{′} ) 〉}{〈 {n}_{A} ( {r}_{1} ) 〉 〈 {n}_{A} ( {r}_{2} ) 〉 〈 {n}_{B} ( {r}_{1}^{′} ) 〉} = {n}_{A}^{2} {n}_{B} {X}_{A} ( {r}_{1} - {r}_{2} ) Y ( {r}_{1} - {r}_{1}^{′} ) Y ( {r}_{2} - {r}_{1}^{′} ) & Equation 11 \\ \frac{\partial {ρ}_{1 , 0}}{\partial t} = \frac{\partial {n}_{A}}{\partial t} \\ \frac{\partial {ρ}_{0 , 1}}{\partial t} = \frac{\partial {n}_{B}}{\partial t} \\ \frac{\partial {ρ}_{2 , 0}}{\partial t} = 2 {n}_{A} {X}_{A} \frac{\partial {n}_{A}}{\partial t} + {n}_{A}^{2} \frac{\partial {X}_{A}}{\partial t} & Equation 12 \\ \frac{\partial {ρ}_{0 , 2}}{\partial t} = 2 {n}_{B} {X}_{B} \frac{\partial {n}_{B}}{\partial t} + {n}_{B}^{2} \frac{\partial {X}_{B}}{\partial t} \\ \frac{\partial {ρ}_{1 , 1}}{\partial t} = {n}_{B} Y \frac{\partial {n}_{A}}{\partial t} + {n}_{A} Y \frac{\partial {n}_{B}}{\partial t} + {n}_{A} {n}_{B} \frac{\partial Y}{\partial t} & \\ \frac{\partial {n}_{A} ( t )}{\partial t} = {k}_{off} {n}_{B} ( t ) - {k}_{on}^{*} {n}_{A} ( t ) + λ {n}_{C} ( t ) + {k}_{cat}^{*} {n}_{B} ( t ) \int σ ( {r}^{′} ) {Y}_{B C} ( {r}^{′} , t ) {d}^{d} {r}^{′} \\ \frac{\partial {n}_{B} ( t )}{\partial t} = {k}_{on}^{*} {n}_{A} ( t ) - {k}_{off} {n}_{B} ( t ) & Equation 12 ’ \\ \frac{\partial {n}_{C} ( t )}{\partial t} = - λ {n}_{C} ( t ) - {k}_{cat}^{*} {n}_{B} ( t ) {n}_{C} ( t ) \int σ ( {r}^{′} ) {Y}_{BC} ( {r}^{′} , t ) {d}^{d} {r}^{′} \\ \frac{\partial {X}_{A} ( r , t )}{\partial t} = 2 {k}_{off} \frac{{n}_{B}}{{n}_{A}} [ {Y}_{AB} ( r , t ) - {X}_{A} ( r , t ) ] + 2 λ \frac{{n}_{C}}{{n}_{A}} [ {Y}_{A C} ( r , t ) - {X}_{A} ( r , t ) ] - 2 {k}_{cat}^{*} \frac{{n}_{B} {n}_{C}}{{n}_{A}} {X}_{A} ( r , t ) \int σ ( {r}^{′} ) {Y}_{B C} ( {r}^{′} , t ) {d}^{d} {r}^{′} + {k}_{cat}^{*} \frac{{n}_{B} {n}_{C}}{{n}_{A}} {Y}_{A C} ( r , t ) \int σ ( {r}^{′} ) {Y}_{BC} ( {r}^{′} , t ) [ {Y}_{AB} ( r + {r}^{′} ) + {Y}_{AB} ( r - {r}^{′} ) ] {d}^{d} {r}^{′} & \\ \frac{\partial {X}_{B} ( r , t )}{\partial t} = 2 {k}_{o n}^{*} \frac{{n}_{A}}{{n}_{B}} [ {Y}_{AB} ( r , t ) - {X}_{B} ( r , t ) ] & \\ \frac{\partial {X}_{C} ( r , t )}{\partial t} = {k}_{cat}^{*} {n}_{B} ( t ) {X}_{C} ( r , t ) \int σ ( {r}^{′} ) {Y}_{BC} ( {r}^{′} , t ) [ 2 - {Y}_{BC} ( {r}^{′} + r , t ) - {Y}_{BC} ( {r}^{′} - r , t ) ] {d}^{d} {r}^{′} & \\ \frac{\partial {Y}_{AB} ( r , t )}{\partial t} = {k}_{off} \frac{{n}_{B}}{{n}_{A}} [ {X}_{B} ( r , t ) - {Y}_{AB} ( r , t ) ] + {k}_{on}^{*} \frac{{n}_{A}}{{n}_{B}} [ {X}_{A} ( r , t ) - {Y}_{AB} ( r , t ) ] + λ \frac{{n}_{C}}{{n}_{A}} [ {Y}_{BC} ( r , t ) - {Y}_{AB} ( r , t ) ] + {k}_{cat}^{*} \frac{{n}_{C}}{{n}_{A}} σ ( r ) {Y}_{BC} ( r , t ) - {k}_{cat}^{*} \frac{{n}_{B} {n}_{C}}{{n}_{A}} {Y}_{AB} ( r , t ) \int σ ( {r}^{′} ) {Y}_{BC} ( {r}^{′} , {t}^{′} ) {d}^{d} {r}^{′} + {k}_{cat}^{*} \frac{{n}_{B} {n}_{C}}{{n}_{A}} {Y}_{BC} ( r , t ) \int σ ( {r}^{′} , t ) {X}_{B} ( r + {r}^{′} , t ) {d}^{d} {r}^{′} & \\ \frac{\partial {Y}_{BC} ( r , t )}{\partial t} = {k}_{o n}^{*} \frac{{n}_{A}}{{n}_{B}} [ {Y}_{A C} ( r , t ) - {Y}_{BC} ( r , t ) ] - {k}_{cat}^{*} σ ( r ) {Y}_{BC} ( r , t ) + {k}_{cat}^{*} {n}_{B} {Y}_{BC} \int σ ( {r}^{′} ) {Y}_{BC} ( {r}^{′} , t ) [ 1 - {X}_{B} ( r - {r}^{′} , t ) ] {d}^{d} {r}^{′} & \\ \frac{\partial {Y}_{A C} ( r , t )}{\partial t} = {k}_{off} \frac{{n}_{B}}{{n}_{A}} [ {Y}_{BC} ( r , t ) - {Y}_{A C} ( r , t ) ] + λ \frac{{n}_{C}}{{n}_{A}} [ {X}_{C} ( r , t ) - {Y}_{A C} ( r , t ) ] + ( 1 - \frac{{n}_{C}}{{n}_{A}} ) {k}_{cat}^{*} {n}_{B} {Y}_{A C} ( r , t ) \int σ ( {r}^{′} ) {Y}_{BC} ( {r}^{′} , t ) {d}^{d} {r}^{′} - {k}_{cat}^{*} {n}_{B} {Y}_{A C} ( r , t ) \int σ ( {r}^{′} ) {Y}_{BC} ( {r}^{′} , t ) {Y}_{AB} ( r + {r}^{′} ) {d}^{d} {r}^{′} + {k}_{cat}^{*} \frac{{n}_{B} {n}_{C}}{{n}_{A}} {X}_{C} ( r , t ) \int σ ( {r}^{′} ) {Y}_{BC} ( {r}^{′} , t ) {Y}_{BC} ( {r}^{′} - r ) {d}^{d} {r}^{′} & \\ \frac{\partial {n}_{A}}{\partial t} = - ( {k}_{o n}^{*} + λ ) {n}_{A} + {k}_{off} {n}_{B} - {n}_{A} {n}_{B} \int μσ ( {r}^{′} ) Y ( {r}^{′} ) {d}^{3} {r}^{′} \\ \frac{\partial {n}_{B}}{\partial t} = {k}_{o n}^{*} {n}_{A} - {k}_{off} {n}_{B} \\ \frac{\partial Y}{\partial t} = \frac{{k}_{o n}^{*} {n}_{A}}{{n}_{B}} ( {X}_{A} - Y ) + \frac{{k}_{off} {n}_{B}}{{n}_{A }} ( {X}_{B} - Y ) - μσ ( r ) Y - {n}_{B} Y \int μσ ( {r}^{′} ) Y ( {r}^{′} ) ( {X}_{B} ( {r}^{′} - r ) - 1 ) {d}^{3} {r}^{′} \\ \frac{\partial {X}_{A}}{\partial t} = \frac{2 {k}_{off} {n}_{B}}{{n}_{B}} ( Y - {X}_{A} ) - {n}_{B} {X}_{A} \int μ σ ( {r}^{′} ) Y ( {r}^{′} ) ( Y ( {r}^{′} - r ) + Y ( {r}^{′} + r ) - 2 ) {d}^{3} {r}^{′} \\ \frac{\partial {X}_{B}}{\partial t} = \frac{2 {k}_{on}^{*} {n}_{A}}{{n}_{B}} ( Y - {X}_{B} ) & Equation 13 \\ \frac{\partial {n}_{A} ( t )}{\partial t} = {k}_{off} {n}_{B} ( t ) - {k}_{o n}^{*} {n}_{A} ( t ) + λ {n}_{C} ( t ) + 4 π {k}_{cat}^{*} {n}_{B} ( t ) {n}_{C} ( t ) {\int }_{0}^{∞} {r}^{′2} σ ( {r}^{′} ) {Y}_{BC} ( {r}^{′} , t ) {dr}^{′} \\ \frac{\partial {n}_{B} ( t )}{\partial t} = {k}_{o n}^{*} {n}_{A} ( t ) - {k}_{off} {n}_{B} ( t ) & Equation 13 ’ \\ \frac{\partial {n}_{C} ( t )}{\partial t} = - λ {n}_{C} ( t ) - 4 π {k}_{cat}^{*} {n}_{B} ( t ) {n}_{C} ( t ) {\int }_{0}^{∞} {r}^{′2} σ ( {r}^{′} ) {Y}_{BC} ( {r}^{′} , t ) {dr}^{′} & \\ \frac{\partial {X}_{A} ( r , t )}{\partial t} = 2 {k}_{off} \frac{{n}_{B}}{{n}_{A }} [ {Y}_{AB} ( r , t ) - {X}_{A} ( r , t ) ] + 2 λ \frac{{n}_{C}}{{n}_{A}} [ {Y}_{A C} ( r , t ) - {X}_{A} ( r , t ) ] - 8 π {k}_{cat}^{*} \frac{{n}_{B} {n}_{C}}{{n}_{A}} {X}_{A} ( r , t ) {\int }_{0}^{∞} {r}^{′2} σ ( {r}^{′} ) {Y}_{BC} ( {r}^{′} , t ) {dr}^{′} + \frac{4 π}{r} {k}_{cat}^{*} \frac{{n}_{B} {n}_{C}}{{n}_{A}} {Y}_{A C} ( r , t ) {\int }_{0}^{∞} {r}^{′} σ ( {r}^{′} ) {Y}_{BC} ( {r}^{′} , t ) {\int }_{\vert r - {r}^{′} \vert}^{\vert r + {r}^{′} \vert} {qY}_{AB} ( q , t ) {dqdr}^{′} & \\ \frac{\partial {X}_{B} ( r , t )}{\partial t} = 2 {k}_{o n}^{*} \frac{{n}_{A}}{{n}_{B}} [ {Y}_{AB} ( r , t ) - {X}_{B} ( r , t ) ] & \\ \frac{\partial {X}_{C} ( r , t )}{\partial t} = \frac{4 π}{r} {k}_{cat}^{*} {n}_{B} ( t ) {X}_{C} ( r , t ) {\int }_{0}^{∞} {r}^{′} σ ( {r}^{′} ) {Y}_{BC} ( {r}^{′} , t ) {\int }_{\vert r - {r}^{′} \vert}^{\vert r + {r}^{′} \vert} q [ 1 - {Y}_{BC} ( q , t ) ] {dqdr}^{′} & \\ \frac{\partial {Y}_{AB} ( r , t )}{\partial t} = {k}_{off} \frac{{n}_{B}}{{n}_{A }} [ {X}_{B} ( r , t ) - {Y}_{AB} ( r , t ) ] + {k}_{o n}^{*} \frac{{n}_{A}}{n} [ {X}_{A} ( r , t ) - {X}_{AB} ( r , t ) ] + λ \frac{{n}_{C}}{{n}_{A}} [ {Y}_{BC} ( r , t ) - {Y}_{AB} ( r , t ) ] + {k}_{cat}^{*} \frac{{n}_{C}}{{n}_{A}} σ ( r ) {Y}_{BC} ( r , t ) - 4 π {k}_{cat}^{*} \frac{{n}_{B} {n}_{C}}{{n}_{A}} {Y}_{AB} ( r , t ) {\int }_{0}^{∞} {r}^{′2} σ ( {r}^{′} ) {Y}_{BC} ( {r}^{′} , t ) {dr}^{′} + \frac{2 π}{r} {k}_{cat}^{*} \frac{{n}_{B} {n}_{C}}{{n}_{A}} {Y}_{BC} ( r , t ) {\int }_{0}^{∞} {r}^{′} σ ( {r}^{′} ) {Y}_{BC} ( {r}^{′} , t ) {\int }_{\vert r - {r}^{′} \vert}^{\vert r + {r}^{′} \vert} {qX}_{B} ( q , t ) {dqdr}^{′} & \\ \frac{\partial {Y}_{BC} ( r , t )}{\partial t} = {k}_{o n}^{*} \frac{{n}_{A}}{{n}_{B}} [ {Y}_{A C} ( r , t ) - {Y}_{BC} ( r , t ) ] - {k}_{cat}^{*} σ ( r ) {Y}_{BC} ( r , t ) + \frac{2 π}{r} {k}_{cat}^{*} {n}_{B} {Y}_{BC} {\int }_{0}^{∞} {r}^{′} σ ( {r}^{′} ) {Y}_{BC} ( {r}^{′} , t ) {\int }_{\vert r - {r}^{′} \vert}^{\vert r + {r}^{′} \vert} q [ 1 - {X}_{B} ( q , t ) ] {dqdr}^{′} & \\ \frac{\partial {Y}_{A C} ( r , t )}{\partial t} = {k}_{off} \frac{{n}_{B}}{{n}_{A}} [ {Y}_{BC} ( r , t ) - {Y}_{A C} ( r , t ) ] + λ \frac{{n}_{C}}{{n}_{A}} [ {X}_{C} ( r , t ) - {Y}_{A C} ( r , t ) ] + 4 π ( 1 - \frac{{n}_{C}}{{n}_{A}} ) {k}_{cat}^{*} {n}_{B} {Y}_{A C} ( r , t ) {\int }_{0}^{∞} {r}^{′2} σ ( {r}^{′} ) {Y}_{BC} ( {r}^{′} , t ) {dr}^{′} - \frac{2 π}{r} {k}_{cat}^{*} {n}_{B} {Y}_{A C} ( r , t ) {\int }_{0}^{∞} {r}^{′} σ ( {r}^{′} ) {Y}_{BC} ( {r}^{′} , t ) {\int }_{\vert r - {r}^{′} \vert}^{\vert r + {r}^{′} \vert} {qY}_{AB} ( q , t ) {dqdr}^{′} + \frac{2 π}{r} {k}_{cat}^{*} \frac{{n}_{B} {n}_{C}}{{n}_{A}} {X}_{C} ( r , t ) {\int }_{0}^{∞} {r}^{′} σ ( {r}^{′} ) {Y}_{BC} ( {r}^{′} , t ) {\int }_{\vert r - {r}^{′} \vert}^{\vert r + {r}^{′} \vert} {qY}_{BC} ( q , t ) {dqdr}^{′} & \\ \begin{matrix}\int G ( {r}^{′} ) {d}^{3} {r}^{′} = {\int }_{0}^{∞} {dr}^{′} {\int }_{0}^{2 π} d φ {\int }_{0}^{π} d {θ}^{′} [ {( {r}^{′} )}^{2} sin ( {θ}^{′} ) G ( {r}^{′} ) ] \\ = 4 π {\int }_{0}^{∞} {dr}^{′} [ {( {r}^{′} )}^{2} G ( {r}^{′} ) ]\end{matrix} & Equation 14 \\ \begin{matrix}\int G ( r - {r}^{′} ) {d}^{3} {r}^{′} = {\int }_{0}^{∞} {dr}^{′} {\int }_{0}^{2 π} d φ {\int }_{0}^{π} d {θ}^{′} \\ [ {( {r}^{′} )}^{2} sin ( {θ}^{′} ) G ( \vert r - {r}^{′} \vert ) ] \\ = \frac{2 π}{r} {\int }_{0}^{∞} {dr}^{′} {\int }_{\vert r - {r}^{′} \vert}^{\vert r + {r}^{′} \vert} dq [ {qr}^{′} G ( q ) ]\end{matrix} & Equation 15 \\ \frac{\partial {n}_{A}}{\partial t} = - ( {p}_{1} + {p}_{5} ) {n}_{A} + {p}_{2} {n}_{B} - 4 {π ( 3 / 2 π )}^{3 / 2} {p}_{3} {n}_{A} {n}_{B} {\int }_{0}^{∞} {dr}^{′} [ {( {r}^{′} )}^{2} {e}^{- \frac{3 {( {r}^{′} )}^{2}}{2}} Y ( {r}^{′} ) ] & Equation 16 \\ \frac{\partial {n}_{B}}{\partial t} = {p}_{1} {n}_{A} - {p}_{2} {n}_{B} & \\ \frac{\partial Y}{\partial t} = \frac{{p}_{1} {n}_{A}}{{n}_{B}} ( {X}_{A} - Y ) + \frac{{p}_{2} {n}_{B}}{{n}_{A}} ( {X}_{B} - Y ) - {( \frac{3}{2 π} )}^{3 / 2} {p}_{4} {e}^{- \frac{3 {r}^{2}}{2}} Y - 2 {π ( \frac{3}{2 π} )}^{3 / 2} {p}_{3} \frac{{n}_{B} Y}{r} ( {\int }_{0}^{∞} {dr}^{′} {\int }_{\vert r - {r}^{′} \vert}^{\vert r + {r}^{′} \vert} dq [ {qr}^{′} {e}^{- \frac{3 {( {r}^{′} )}^{2}}{2}} Y ( {r}^{′} ) ( {X}_{B} ( q ) - 1 ) ] ) & \\ \frac{\partial {X}_{A}}{\partial t} = \frac{2 {p}_{2} {n}_{B}}{{n}_{A}} ( Y - {X}_{A} ) - 4 {π ( \frac{3}{2 π} )}^{3 / 2} {p}_{3} \frac{{n}_{B} {X}_{A}}{r} ( {\int }_{0}^{∞} {dr}^{′} {\int }_{\vert r - {r}^{′} \vert}^{\vert r + {r}^{′} \vert} dq [ {qr}^{′} {e}^{- \frac{3 {( {r}^{′} )}^{2}}{2}} Y ( {r}^{′} ) ( Y ( q ) - 1 ) ] ) & \\ \frac{\partial {X}_{B}}{\partial t} = \frac{2 {p}_{1} {n}_{A}}{{n}_{B}} ( Y - {X}_{B} ) & \\ \frac{\partial {n}_{A} ( t )}{\partial t} = {k}_{off} {n}_{B} ( t ) - {k}_{o n}^{*} {n}_{A} ( t ) + λ {n}_{C} ( t ) + 4 {π ( \frac{3}{2 π} )}^{3 / 2} {k}_{cat}^{*} {C}_{T} {n}_{B} ( t ) {n}_{C} ( t ) {\int }_{0}^{∞} {r}^{′2} {e}^{- \frac{3 {r}^{′2}}{2}} {Y}_{BC} ( {r}^{′} , t ) {dr}^{′} \\ \frac{\partial {n}_{B} ( t )}{\partial t} = {k}_{o n}^{*} {n}_{A} ( t ) - {k}_{off} {n}_{B} ( t ) & Equation 16 ’ \\ \frac{\partial {n}_{C} ( t )}{\partial t} = - λ {n}_{C} ( t ) - 4 {π ( \frac{3}{2 π} )}^{3 / 2} {k}_{cat}^{*} {C}_{T} {n}_{B} {n}_{C} ( t ) {\int }_{0}^{∞} {r}^{′2} {e}^{- \frac{3 {r}^{′2}}{2}} {Y}_{BC} ( {r}^{′} , t ) {dr}^{′} & \\ \frac{\partial {X}_{A} ( r , t )}{\partial t} = 2 {k}_{off} \frac{{n}_{B}}{{n}_{A}} [ {Y}_{AB} ( r , t ) - {X}_{A} ( r , t ) ] + 2 λ \frac{{n}_{C}}{{n}_{A}} [ {Y}_{A C} ( r , t ) - {X}_{A} ( r , t ) ] - 8 {π ( \frac{3}{2 π} )}^{3 / 2} {k}_{cat}^{*} {C}_{T} \frac{{n}_{B} {n}_{C}}{{n}_{A}} {X}_{A} ( r , t ) {\int }_{0}^{∞} {r}^{′2} {e}^{- \frac{3 {r}^{′2}}{2}} {Y}_{BC} ( {r}^{′} , t ) {dr}^{′} + \frac{4 π}{r} {( \frac{3}{2 π} )}^{3 / 2} {k}_{cat}^{*} {C}_{T} \frac{{n}_{B} {n}_{C}}{{n}_{A}} {Y}_{A C} ( r , t ) {\int }_{0}^{∞} {r}^{′} {e}^{- \frac{3 {r}^{′2}}{2}} {Y}_{BC} ( {r}^{′} , t ) {dr}^{′} + \frac{4 π}{r} {( \frac{3}{2 π} )}^{3 / 2} {k}_{cat}^{*} {C}_{T} \frac{{n}_{B} {n}_{C}}{{n}_{A}} {Y}_{A C} ( r , t ) {\int }_{0}^{∞} {r}^{′} {e}^{- \frac{3 {r}^{′2}}{2}} {Y}_{BC} ( {r}^{′} , t ) \cdot {\int }_{\vert r - {r}^{′} \vert}^{\vert r + {r}^{′} \vert} {qY}_{AB} ( q , t ) {dqdr}^{′} & \\ \frac{\partial {X}_{B} ( r , t )}{\partial t} = 2 {k}_{on}^{*} \frac{{n}_{A}}{{n}_{B}} [ {Y}_{AB} ( r , t ) - {X}_{B} ( r , t ) ] & \\ \frac{\partial {X}_{C} ( r , t )}{\partial t} = \frac{4 π}{r} {( \frac{3}{2 π} )}^{3 / 2} {k}_{cat}^{*} {C}_{T} {n}_{B} ( t ) {X}_{C} ( r , t ) {\int }_{0}^{∞} {r}^{′} {e}^{- \frac{3 {r}^{′2}}{2}} {Y}_{BC} ( {r}^{′} , t ) \cdot {\int }_{\vert r - {r}^{′} \vert}^{\vert r + {r}^{′} \vert} q [ 1 - {Y}_{BC} ( q , t ) ] {dqdr}^{′} & \\ \frac{\partial {Y}_{AB} ( r , t )}{\partial t} = {k}_{off} \frac{{n}_{B}}{{n}_{A}} [ {X}_{B} ( r , t ) - {Y}_{AB} ( r , t ) ] + {k}_{on}^{*} \frac{{n}_{A}}{{n}_{B}} [ {X}_{A} ( r , t ) - {Y}_{AB} ( r , t ) ] + λ \frac{{n}_{C}}{{n}_{A}} [ {Y}_{BC} ( r , t ) - {Y}_{AB} ( r , t ) ] + {( \frac{3}{2 π} )}^{3 / 2} \frac{{k}_{cat}^{*}}{{L}^{3}} \frac{{n}_{C}}{{n}_{A}} {e}^{- \frac{3 {r}^{′2}}{2}} {Y}_{BC} ( r , t ) - 4 {π ( \frac{3}{2 π} )}^{3 / 2} {k}_{cat}^{*} {C}_{T} \frac{{n}_{B} {n}_{C}}{{n}_{A}} {Y}_{AB} ( r , t ) {\int }_{0}^{∞} {r}^{′2} {e}^{- \frac{3 {r}^{′2}}{2}} {Y}_{BC} ( {r}^{′} , t ) {dr}^{′} + \frac{2 π}{r} {( \frac{3}{2 π} )}^{3 / 2} {k}_{cat}^{*} {C}_{T} \frac{{n}_{B} {n}_{C}}{{n}_{A}} {Y}_{BC} ( r , t ) {\int }_{0}^{∞} {r}^{′} {e}^{- \frac{3 {r}^{′2}}{2}} {Y}_{BC} ( {r}^{′} , t ) \cdot {\int }_{\vert r - {r}^{′} \vert}^{\vert r + {r}^{′} \vert} {qX}_{B} ( q , t ) {dqdr}^{′} & \\ \frac{\partial {Y}_{BC} ( r , t )}{\partial t} = {k}_{on}^{*} \frac{{n}_{A}}{{n}_{B }} [ {Y}_{A C} ( r , t ) - {Y}_{BC} ( r , t ) ] - {( \frac{3}{2 π} )}^{3 / 2} \frac{{k}_{cat}^{*}}{{L}^{3}} {e}^{- \frac{3 {r}^{2}}{2}} {Y}_{BC} ( r , t ) + \frac{2 π}{r} {( \frac{3}{2 π} )}^{3 / 2} {k}_{cat}^{*} {C}_{T} {n}_{B} {Y}_{BC} {\int }_{0}^{∞} {r}^{′} {e}^{- \frac{3 {r}^{′2}}{2}} {Y}_{BC} ( {r}^{′} , t ) \cdot {\int }_{\vert r - {r}^{′} \vert}^{\vert r + {r}^{′} \vert} q [ 1 - {X}_{B} ( q , t ) ] {dqdr}^{′} & \\ \frac{\partial {Y}_{A C} ( r , t )}{\partial t} = {k}_{off} \frac{{n}_{B}}{{n}_{A}} [ {Y}_{BC} ( r , t ) - {Y}_{A C} ( r , t ) ] + λ \frac{{n}_{C}}{{n}_{B}} [ {X}_{C} ( r , t ) - {Y}_{A C} ( r , t ) ] + 4 π {( \frac{3}{2 π} )}^{3 / 2} ( 1 - \frac{{n}_{C}}{{n}_{A}} ) {k}_{cat}^{*} {C}_{T} {n}_{B} {Y}_{A C} ( r , t ) {\int }_{0}^{∞} {r}^{′2} {e}^{- \frac{3 {r}^{′2}}{2}} {Y}_{BC} ( {r}^{′} , t ) {dr}^{′} - \frac{2 π}{r} {( \frac{3}{2 π} )}^{3 / 2} {k}_{cat}^{*} {C}_{T} {n}_{B} {Y}_{A C} ( r , t ) {\int }_{0}^{∞} {r}^{′} {e}^{- \frac{3 {r}^{′2}}{2}} {Y}_{BC} ( {r}^{′} , t ) \cdot {\int }_{\vert r - {r}^{′} \vert}^{\vert r + {r}^{′} \vert} {qY}_{AB} ( q , t ) {dqdr}^{′} + \frac{2 π}{r} {( \frac{3}{2 π} )}^{3 / 2} {k}_{cat}^{*} {C}_{T} \frac{{n}_{B} {n}_{C}}{{n}_{A}} {X}_{C} ( r , t ) {\int }_{0}^{∞} {r}^{′ } {e}^{- \frac{3 {r}^{′ 2}}{2}} {Y}_{BC} ( {r}^{′} , t ) \cdot {\int }_{\vert r - {r}^{′} \vert}^{\vert r + {r}^{′} \vert} {qY}_{BC} ( q , t ) {dqdr}^{′} & \end{matrix}$$

1. A computer-implemented method of determining characteristics of enzyme catalysis, comprising:
receiving, from a surface plasmon resonance (SPR) instrument, SPR data indicative of binding of an enzyme and a substrate;
determining one or more characteristics of the enzyme catalysis based on a multi-centre particle density (MPD) model and the SPR data.

2. The method of claim 1, wherein determining the one or more characteristics comprises fitting the MPD model to the SPR data.

3. The method of claim 1, wherein one or more characteristics comprise one or more of catalytic rate (k*_{cat}), association (kₒₙ) and dissociation (k_{off}) of the enzyme.

4. The method of claim 1, wherein the one or more characteristics comprise reach length (L).

5. The method of claim 1, wherein the determining comprises determining catalytic rate (k_{cat}), association (kₒₙ) and dissociation (k_{off}) of the enzyme; and reach length (L).

6. The method of claim 1, wherein the determining the one or more characteristics of the enzyme catalysis comprises fitting the multi-centre particle density (MPD) model to the SPR data.

7. The method of claim 1, wherein the MPD model comprises a system of multi-centre particle distributions (MPD).

8. The method of claim 7, wherein the system of MPDs comprises:
$$\frac{\partial {n}_{A}}{\partial t} = - ( {p}_{1} + {p}_{5} ) {n}_{A} + {p}_{2} {n}_{B} - 4 {π ( 3 / 2 π )}^{3 / 2} {p}_{3} {n}_{A} {n}_{B} {\int }_{0}^{∞} {dr}^{′} [ {( {r}^{′} )}^{2} {e}^{- \frac{3 {( {r}^{′} )}^{2}}{2}} Y ( {r}^{′} ) ] \\ \frac{\partial {n}_{B}}{\partial t} = {p}_{1} {n}_{A} - {p}_{2} {n}_{B} \\ \frac{\partial Y}{\partial t} = \frac{{p}_{1} {n}_{A}}{{n}_{B}} ( {X}_{A} - Y ) + \frac{{p}_{2} {n}_{B}}{{n}_{A}} ( {X}_{B} - Y ) - {( \frac{3}{2 π} )}^{3 / 2} {p}_{4} {e}^{- \frac{3 {r}^{2}}{2}} Y - 2 {π ( \frac{3}{2 π} )}^{3 / 2} {p}_{3} \frac{{n}_{B} Y}{r} ( {\int }_{0}^{∞} {dr}^{′} {\int }_{\vert r - {r}^{′} \vert}^{\vert r + {r}^{′} \vert} dq [ {qr}^{′} {e}^{- \frac{3 {( {r}^{′} )}^{2}}{2}} Y ( {r}^{′} ) ( {X}_{B} ( q ) - 1 ) ] ) \\ \frac{\partial {X}_{A}}{\partial t} = \frac{2 {p}_{2} {n}_{B}}{{n}_{A}} ( Y - {X}_{A} ) - 4 {π ( \frac{3}{2 π} )}^{3 / 2} {p}_{3} \frac{{n}_{B} {X}_{A}}{r} ( {\int }_{0}^{∞} {dr}^{′} {\int }_{\vert r - {r}^{′} \vert}^{\vert r + {r}^{′} \vert} dq [ {qr}^{′} {e}^{- \frac{3 {( {r}^{′} )}^{2}}{2}} Y ( {r}^{′} ) ( Y ( q ) - 1 ) ] ) \\ \frac{\partial {X}_{B}}{\partial t} = \frac{2 {p}_{1} {n}_{A}}{{n}_{B}} ( Y - {X}_{B} )$$
Where
A is a phosphorylated substrate distributed randomly in space,
B is an enzyme,
n_{A} is a concentration of A,
n_{B} is a concentration of B,
X_{A} is an autocorrelation function for A,
X_{B} is an autocorrelation function for B,
Y is a pair correlation function between A and B,
p₁, p₂, p₃, p₄, and p₅ are fitting parameters,
r is the position of A,
r′ is the position of B,
q=(r′)²+r²−2rr′ cos(θ).

9. The method of claim 7, wherein each characteristic of the enzyme catalysis is associated with a respective MPD.

10. The method of claim 1, wherein the MPD model comprises a multi-centre particle distribution system of coupled partial differential equations.

11. The method of claim 9, wherein the system of coupled partial differential equations comprises:
$$\frac{\partial {ρ}_{1 , 0}}{\partial t} = - ( {k}_{o n}^{*} + λ ) {ρ}_{1 , 0} + {k}_{off} {ρ}_{0 , 1} - \int μ σ ( {r}_{1} - {r}_{1}^{′} ) {ρ}_{1 , 1} {d}^{3} {r}_{1}^{′}$$
$$\frac{\partial {ρ}_{0 , 1}}{\partial t} = {k}_{o n}^{*} {ρ}_{1 , 0} - {k}_{off} {ρ}_{0 , 1}$$
$$\frac{\partial {ρ}_{1 , 1}}{\partial t} = - ( {k}_{o n}^{*} + {k}_{off} + λ ) {ρ}_{1 , 1} + {k}_{o n}^{*} {ρ}_{2 , 0} + {k}_{off} {ρ}_{0 , 2} - μ σ ( {r}_{1} - {r}_{1}^{′} ) {ρ}_{1 , 1} - \int μ σ ( {r}_{1} - {r}_{2}^{′} ) {ρ}_{1 , 2} {d}^{3} {r}_{2}^{′}$$
$$\frac{\partial {ρ}_{2 , 0}}{\partial t} = 2 {k}_{off} {ρ}_{1 , 1} - 2 ( {k}_{on}^{*} + λ ) {ρ}_{2 , 0} - \int μ σ ( {r}_{1} - {r}_{1}^{′} ) {ρ}_{2 , 1} {d}^{3} {r}_{1}^{′} - \int μ σ ( {r}_{2} - {r}_{1}^{′} ) {ρ}_{2 , 1} {d}^{3} {r}_{1}^{′}$$
$$\frac{\partial {ρ}_{0 , 2}}{\partial t} = 2 {k}_{on}^{*} {ρ}_{1 , 1} - 2 {k}_{off} {ρ}_{0 , 2}$$
Where
ρ is the multi-centre particle density,
kₒₙ* is the first-order on-rate or association rate,
k_{off} is the off-rate or dissociation rate,
μ is a surface catalytic rate,
λ is a solution dephosphorylation rate,
σ is a concentration of the substrate, and
r is a position of the polymer tip.

12. The method of claim 1, wherein

13. The method of claim 1, wherein
$$\frac{\partial {ρ}_{m , {m}^{′}}}{\partial t} = - {\sum }_{i = 1}^{m} {\sum }_{j = 1}^{{m}^{′}} μ σ ( {r}_{i} - {r}_{j}^{′} ) {ρ}_{m , {m}^{′}} - {\sum }_{i = 1}^{m} \int μσ ( {r}_{i} - {r}_{{m}^{′} + 1}^{′} ) {ρ}_{m , {m}^{′} + 1} {d}^{3} {r}_{{m}^{′} + 1}^{′} + {k}_{o n}^{*} ( {m}^{′} {ρ}_{m + 1 , {m}^{′} - 1} ) + {k}_{off} ( m {ρ}_{m - 1 , {m}^{′} + 1} ) - ( λ + {k}_{o n}^{*} ) ( m {ρ}_{m , {m}^{′}} ) - {k}_{off} ( {m}^{′} {ρ}_{m , {m}^{′}} )$$
$$and$$
$${ρ}_{m , {m}^{′}} = 〈 {\prod }_{i = 1}^{m} {n}_{A} ( {r}_{i} , t ) {\prod }_{j = 1}^{{m}^{′}} {n}_{B} ( {r}_{j}^{′} , t ) 〉$$
Where
A is a phosphorylated substrate distributed randomly in space,
B is an enzyme,
ρ is the multi-centre particle density,
μ is a surface catalytic rate,
σ is a concentration of the substrate,
kₒₙ* is the first-order on-rate or association rate,
k_{off} is the off-rate or dissociation rate,
rᵢ is a position of the ith A state,
r′ⱼ is a position of the jth B state,
n_{A} is a concentration of A,
n_{B} is a concentration of B,
m is the number of A states, and
m′ is the number of B states.

14. An apparatus for determining characteristics of enzyme catalysis, comprising:
a memory;
an interface for receiving surface plasmon resonance (SPR) data from an SPR instrument indicative of binding of the enzyme and a substrate, and storing the SPR data in a memory;
a processor communicably coupled to the memory, wherein the processor is arranged, in use, to determine one or more characteristics of the enzyme catalysis based on a multi-centre particle density (MPD) model stored in the memory and the SPR data.

15. The apparatus of claim 14, wherein the processor is arranged to perform a method according to claim 2.

16. Computer software which, when executed by a computer, is arranged to perform a method according to claim 1.

17. A computer-readable medium storing computer software as claimed in claim 16.

18. The computer readable medium of claim 17, wherein the computer software is tangibly stored on the computer-readable medium.

19. A system, comprising:
the apparatus of claim 14; and
a surface plasmon resonance (SPR) instrument communicably coupled to the apparatus to, in use, provide SPR data indicative of indicative of binding of an enzyme and a substrate.
